(19) United States
(12) Patent Application Publication
(10) Pub. No.: US 2025/0337280 A1
(43) Pub. Date: Oct. 30, 2025

(54) WIRELESS CHARGING FOR A WEARABLE DEVICE

(71) Applicant: Oura Health Oy, Oulu (FI)

(72) Inventor: Reetta Sofia Kuonanoja, Oulu (FI)

(21) Appl. No.: 18/972,512

(22) Filed: Dec. 6, 2024

Related U.S. Application Data
(60) Provisional application No. 63/640,787, filed on Apr. 30, 2024.

(51) Int. Cl. H02J 50/12; A61B 5/024; H02J 7/00; H02J 50/00; H02J 50/40

(52) U.S. Cl. CPC H02J 50/12; A61B 5/02427; H02J 7/0044; H02J 50/005; H02J 50/402; A61B 2560/0214

(57) ABSTRACT

Methods, systems, and devices for charging a wearable device are described. A charging system may include charging station with a resonant coil configured to generate an electromagnetic field based at least in part on a first alternating current. The charging system may include a wearable device with a load coil inductively coupled with the resonant coil of the charging station, configured to have a same resonant frequency as the resonant coil, and configured to generate a second alternating current based at least in part on the electromagnetic field generated by the resonant coil.

### CROSS REFERENCE

The present Application for Patent claims priority to U.S. Patent Application No. 63/640,787 by Kuonanoja, entitled “WIRELESS CHARGING FOR A WEARABLE DEVICE,” filed Apr. 30, 2024, which is assigned to the assignee hereof, and which is expressly incorporated by reference herein.

### FIELD OF TECHNOLOGY

The following relates to wearable devices and data processing, including wireless charging for a wearable device.

### BACKGROUND

A wearable device configured to collect biometric data from a user may have a rechargeable battery. Improved techniques for wirelessly charging the battery of a wearable device may be desired.

### BRIEF DESCRIPTION OF THE DRAWINGS

FIG. 1 illustrates an example of a system that supports wireless charging for a wearable device in accordance with aspects of the present disclosure.

FIG. 2 illustrates an example of a system that supports wireless charging for a wearable device in accordance with aspects of the present disclosure.

FIG. 3A shows an example of a charging system that supports wireless charging for a wearable device in accordance with aspects of the present disclosure.

FIG. 3B shows an example of a charging system that supports wireless charging for a wearable device in accordance with aspects of the present disclosure.

FIG. 4 shows an example of a charging system that supports wireless charging for a wearable device in accordance with aspects of the present disclosure.

FIG. 5 shows an example of a charging system that supports wireless charging for a wearable device in accordance with aspects of the present disclosure.

FIG. 6 shows an example of a charging system that supports wireless charging for a wearable device in accordance with aspects of the present disclosure.

FIG. 7 shows a block diagram of an apparatus that supports wireless charging for a wearable device in accordance with aspects of the present disclosure.

FIG. 8 shows a block diagram of a wearable device manager that supports wireless charging for a wearable device in accordance with aspects of the present disclosure.

FIG. 9 shows a diagram of a system including a device that supports wireless charging for a wearable device in accordance with aspects of the present disclosure.

FIG. 10 shows a flowchart illustrating methods that support wireless charging for a wearable device in accordance with aspects of the present disclosure.

### DETAILED DESCRIPTION

A wearable device configured to collect biometric data from a user may draw power from a rechargeable battery. To wirelessly charge the battery, an induction charging technique may be used in which an electromagnetic field generated by a source coil of a charging station is used to stimulate an alternating current on a load coil of the wearable device. The efficiency of power transfer using the inductive charging technique may be proportionally related to the size of the load coil, and thus may be limited by the body of the wearable device (which limits the size of the load coil). Further, heat dissipation may increase with the size (e.g., loop diameter) of the load coil. Additionally, the inductive charging technique may be susceptible to coil misalignment between the source coil and load coil in that the power transferred between the coils may decrease with the severity of coil misalignment.

To address these issues, among others, a charging system that uses a resonant charging technique may be implemented to charge a wearable device, where charging a wearable device may refer to charging one or more batteries of the wearable device. A charging station of the charging system may include a resonant coil that is configured to have the same (or a similar) resonant frequency as a load coil of the wearable device. The resonant coil may be inductively coupled with the load coil to facilitate wireless power transfer from the charging station to the wearable device. In some cases, the resonant coil may be in addition to a source coil of the charging system. In such cases, the resonant coil may be inductively coupled with both the load coil and the source coil.

Use of the resonant coil may enable (e.g., without sacrificing power transfer efficiency) use of a load coil with a shorter overall length (relative to other designs for other techniques), which in turn may allow for smaller-bodied wearable devices and lower heat dissipation (e.g., due to lower load coil resistance). The resonant charging technique may also be less susceptible to coil misalignment relative to other wireless charging techniques.

Aspects of the disclosure are initially described in the context of systems supporting physiological data collection from users via wearable devices. Additional aspects of the disclosure are described with reference to wireless charging systems. Aspects of the disclosure are further illustrated by and described with reference to apparatus diagrams, system diagrams, and flowcharts that relate to wireless charging for a wearable device.

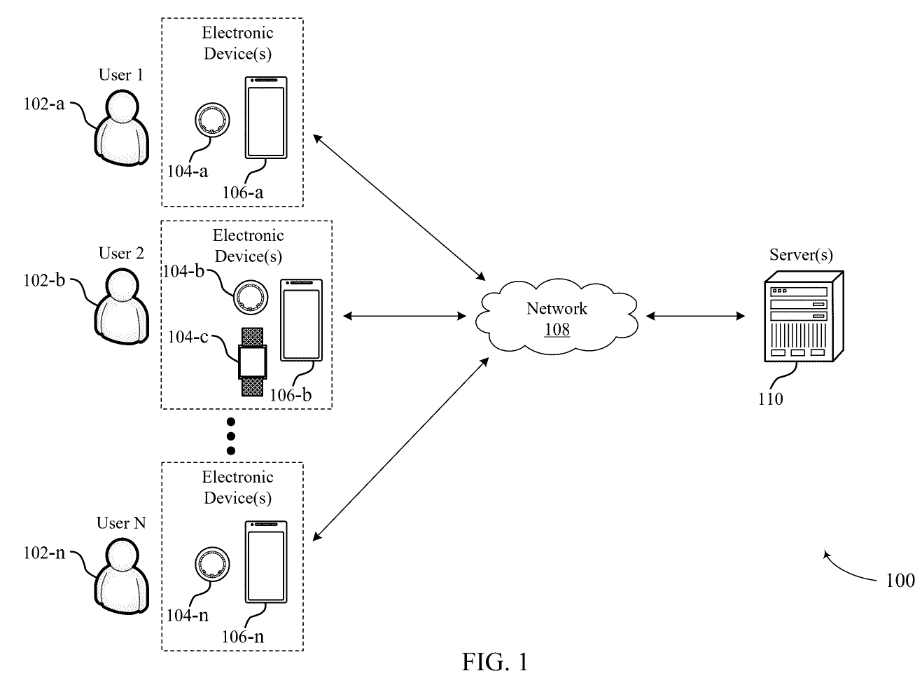

FIG. 1 illustrates an example of a system 100 that supports wireless charging for a wearable device in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the car, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, blood oxygen saturation (SpO2), blood sugar levels (e.g., glucose metrics), and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters. For example, as illustrated in FIG. 1, a first user 102-a (User 1) may operate,

or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols. Moreover, in some cases, the wearable device 104 and the user device 106 may be included within (or make up) the same device. For example, in some cases, the wearable device 104 may be configured to execute an application associated with the wearable device 104, and may be configured to display data via a GUI.

In some implementations, the rings 104 (e.g., wearable devices 104) of the system 100 may be configured to collect physiological data from the respective users 102 based on arterial blood flow within the user's finger. In particular, a ring 104 may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g. in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

A wearable device 104 may be configured to wirelessly charge from a charging station using an inductive resonant charging technique. For example, the wearable device 104 may include a load coil that is inductively coupled with a resonant coil of the charging station such that an alternating current is generated on the load coil in response to an electromagnetic field generated by the resonant coil. To improve power transfer efficiency, the load coil and the resonant coil may be configured with the same (or similar) resonant frequency, where the resonant frequency of a coil refers to the frequency at which the coil exhibits a maximum (or near-maximum) oscillatory response.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

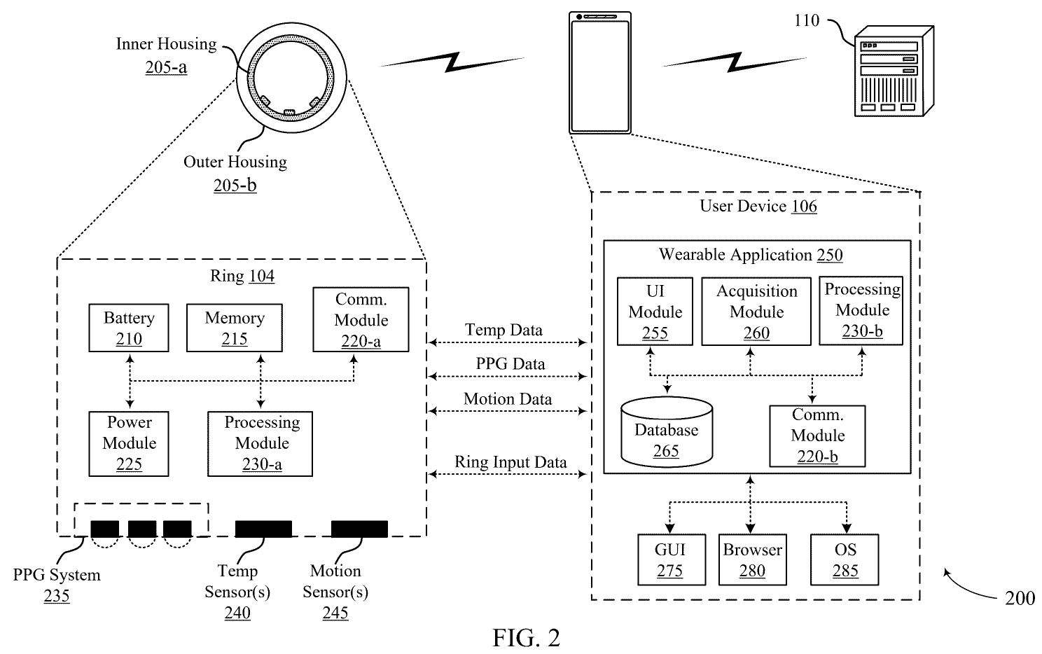

FIG. 2 illustrates an example of a system 200 that supports wireless charging for a wearable device in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the inner housing 205-a, the outer housing 205-b, or both, may include a curved profile/surface. In particular, the housing 205 may exhibit any curved or “circumferential” profile, including a circular profile, an elliptical profile, and the like. Moreover, in some cases, the inner housing 205-a, the outer housing 205-b, or both, may include both curved (e.g., “circumferential”) and flat/planar portions. For the purposes of the present disclosure, the term “circumferential” may be used interchangeably with the term “curved” to refer to circular-shaped, elliptical-shaped, or other curved-shaped profile.

In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using adhesives, wraps, clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during charging, and under voltage during discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BM1160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like.

The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

In some cases, the wearable device 104 and the user device 106 may be included within (or make up) the same device. For example, in some cases, the wearable device 104 may be configured to execute the wearable application 250, and may be configured to display data via the GUI 275.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some examples, the battery 210 may be wireless charged using an inductive resonant charging technique as described herein. For example, the battery 210 may be charged by an alternating current that has been rectified into a direct current by a rectifier of the ring 104. The alternating current may be generated by a load coil of the ring 104 that is inductively coupled with a resonant coil of a charging station. The alternating current may be generated in response to (e.g., based on) an electromagnetic field generated by the resonant coil, which in turn may be generated by an alternating current on a source coil of the charging station. Use of the inductive resonant charging technique may enable (without sacrificing power transfer efficiency) use of a load coil with a shorter overall length (relative to other designs for other wireless charging techniques), which in turn may allow for smaller-bodied wearable devices and lower heat dissipation. The resonant charging technique may also be less susceptible to coil misalignment relative to other wireless charging techniques.

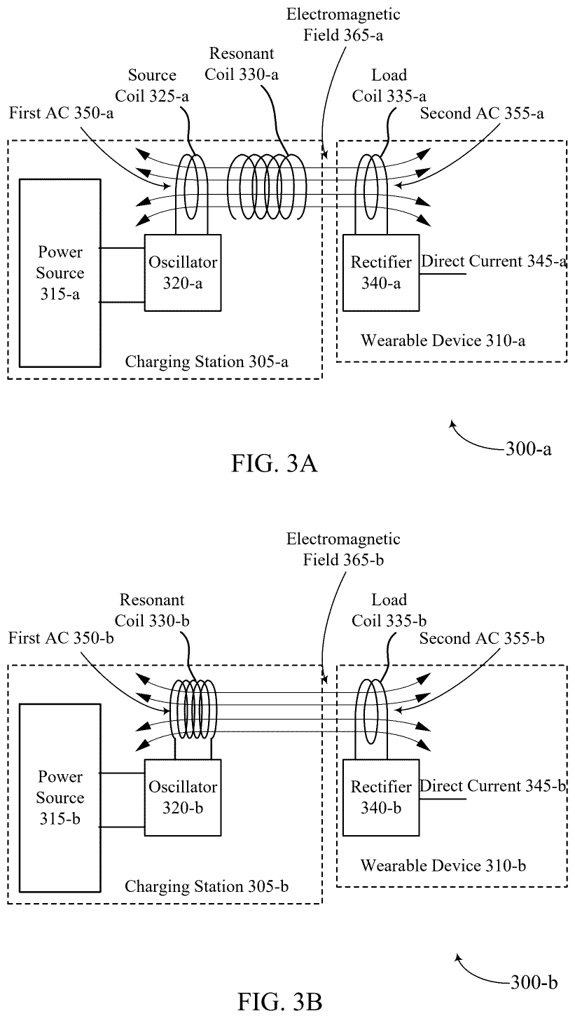

FIG. 3A shows an example of a charging system 300-a that supports wireless charging for a wearable device in accordance with aspects of the present disclosure. The charging system 300-a may include a charging station 305-a and a wearable device 310-a that uses an inductive resonant charging technique to charge from the charging station 305-a. The inductive resonant charging technique may involve one or more coils, which may be loops (e.g., wires) of a conductive material. In some examples, the one or more coils may be inductors.

The charging station 305-a may include an oscillator 320-a that is physically coupled with a power source 315-a and a source coil 325-a. Two components may be referred to as being physically coupled if the two components are in direct contact with (e.g., are touching) each other. The oscillator 320-a may be configured to generate a first alternating current (AC) 350-a on the source coil 325-a using a voltage, current, or both, supplied by the power source 315-a. The source coil 325-a may be configured to generate an electromagnetic field based on (e.g., responsive to) the first alternating current 350-a.

The resonant coil 330-a may be inductively coupled with the source coil 325-a and the load coil 335-a. Two components may be referred to as being inductively coupled if they are configured such that a change in current through one component induces a voltage across the other component through electromagnetic induction. In some examples, the resonant coil 330-a may be physically isolated from the source coil 325-a and the load coil 335-a. Two components may be referred to as being physically isolated if the two components are not in direct contact with (e.g., are not touching) each other.

The resonant coil 330-a may be configured to generate an electromagnetic field 365-a based on (e.g., responsive to) the alternating current on the source coil 325-a. For example, the resonant coil 330-a may be configured to generate an electromagnetic field 365-a at a resonant frequency of the resonant coil 330-a based at least in part on an electromagnetic field generated by the first alternating current 350-a on the source coil 325-a. The source coil 325-a may be configured with the same resonant frequency as the resonant coil 330-a. In some examples, the resonant frequency of the resonant coil 330-a may be based on (e.g., a function of) the intrinsic inductance and the intrinsic capacitance of the resonant coil 330-a. In some examples, the resonant frequency of the resonant coil 330-a may be based on (e.g., a function of) the intrinsic inductance of the resonant coil 330-a and the capacitance of one or more capacitor(s), which may be configured in parallel with the resonant coil 330-a.

The load coil 335-a may be inductive coupled with the resonant coil 330-a and may be physically coupled with the rectifier 340-a. The load coil 335-a may be configured with the same resonant frequency as the resonant coil 330-a. For example, the load coil 335-a may have a first resonant frequency that is equal to (or within a threshold range of) a second resonant frequency of the resonant coil 330-a. Put another way, the resonant frequencies of the source coil 325-a and the load coil 335-a may be within the resonant bandwidth of the resonant coil 330-a, where the resonant bandwidth of the resonant coil 330-a is the range of frequencies around the resonant frequency where the circuit will respond with a threshold level of amplitude. The load coil 335-a may be configured to generate a second alternating current 355-a based on (e.g., responsive to) the electromagnetic field 365-a generated by the resonant coil 330-a.

Due to the configuration of the charging station 305-a and the wearable device 310-a, which may be designed as described herein to ensure close proximity between the load coil 335-a and the resonant coil 330-a, the load coil 335-a may be inductively coupled with the resonant coil 330-a on the charging station 305-a rather than being inductively coupled with a resonant coil on the wearable device 310-a. Thus a second resonant coil on the wearable device 310-a (e.g., between the resonant coil 330-a and the load coil 335-a) may be omitted, which may reduce the amount of space consumed by coils on the wearable device 310-a, among other advantages.

The rectifier 340-a may be configured to receive the second alternating current 355-a from the load coil 335-a and to rectify the second alternating current 355-a into a direct current 345-a that can be used to charge a battery of the wearable device 310-a.

Thus, the wearable device 310-a may wirelessly charge from the charging station 305-a using the inductive resonant charging technique.

FIG. 3B shows an example of a charging system 300-b that supports wireless charging for a wearable device in accordance with aspects of the present disclosure. The charging system 300-b may include a charging station 305-b and a wearable device 310-b that uses an inductive resonant charging technique to charge from the charging station 305-b. The inductive resonant charging technique may involve one or more coils, which may be loops (e.g., wires) of a conductive material. In some examples, the one or more coils may be inductors.

The charging station 305-b may include an oscillator 320-b that is coupled (e.g., physically, electrically, or both) with a power source 315-b and a resonant coil 330-b. The oscillator 320-b may be configured to generate a first alternating current (AC) 350-b on the resonant coil 330-b using a voltage, current, or both, supplied by the power source 315-b. The resonant coil 330-b may be configured to generate an electromagnetic field based on (e.g., responsive to) the first alternating current 350-b.

The resonant coil 330-b may be inductively coupled with the load coil 335-b of the wearable device 310-b (e.g., when the wearable device 310-b is within a proximity threshold to the charger station 305-b). In some examples, the resonant coil 330-b may be physically isolated from the load coil 335-b. The resonant coil 330-b may be configured to generate an electromagnetic field 365-b based on (e.g., responsive to) the alternating current. For example, the resonant coil 330-b may be configured to generate an electromagnetic field 365-b at a resonant frequency of the resonant coil 330-b based at least in part on an electromagnetic field generated by the first alternating current 350-b. In some examples, the resonant frequency of the resonant coil 330-b may be based on (e.g., a function of) the intrinsic inductance and the intrinsic capacitance of the resonant coil 330-b. In some examples, the resonant frequency of the resonant coil 330-b may be based on (e.g., a function of) the intrinsic inductance of the resonant coil 330-b and the capacitance of one or more capacitor(s), which may be configured in parallel with the resonant coil 330-b.

The load coil 335-b may be inductive coupled with the resonant coil 330-b and may be physically coupled with the rectifier 340-b. The load coil 335-b may be configured with the same resonant frequency as the resonant coil 330-b. For example, the load coil 335-b may have a first resonant frequency that is equal to (or within a threshold range of) a second resonant frequency of the resonant coil 330-b. Put another way, the resonant frequencies of the resonant coil 330-b and the load coil 335-b may be within the resonant bandwidth of the resonant coil 330-b, where the resonant bandwidth of the resonant coil 330-b is the range of frequencies around the resonant frequency where the circuit will respond with a threshold level of amplitude.

Due to the configuration of the charging station 305-b and the wearable device 310-b, which may be designed as described herein to ensure close proximity between the load coil 335-b and the resonant coil 330-b, a dedicated source coil on the charging station 305-b (e.g., between the resonant coil 330-b and the oscillator 320-b, as shown in FIG. 3A) may be omitted, which may reduce the amount of space consumed by coils on the charging station 305-b, reduce the manufacturing costs, among other advantages.

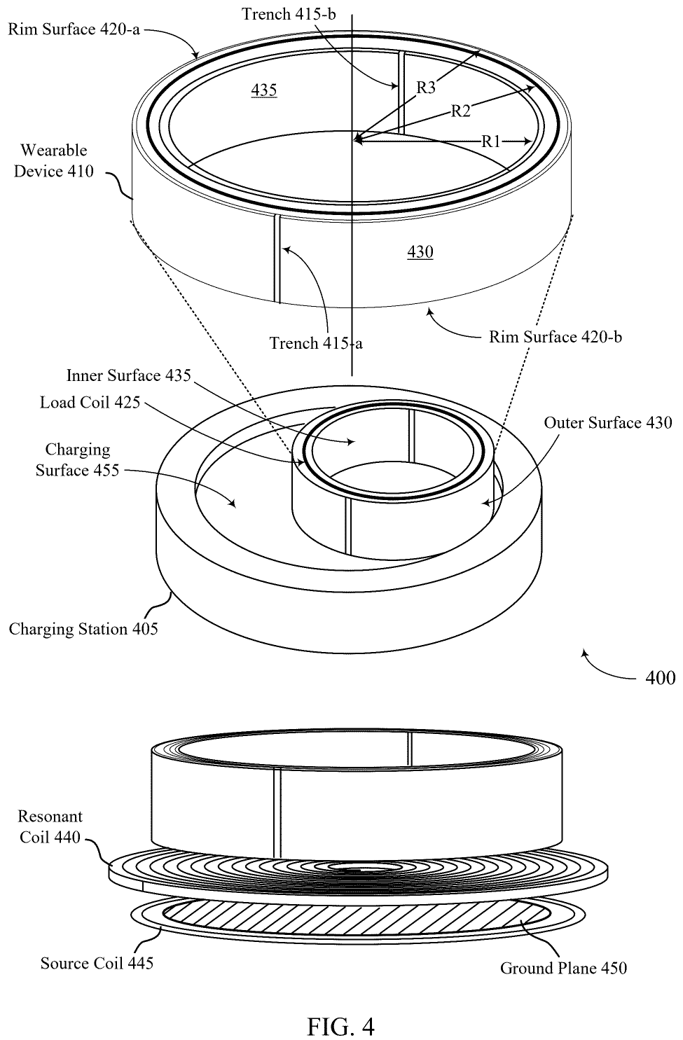

FIG. 4 shows an example of a charging system 400 that supports wireless charging for a wearable device in accordance with aspects of the present disclosure. The charging system 400 may be an example of the charging system 300 as described with reference to FIG. 3. The charging system may include a charging station 405 and a wearable device 410, which may be a wearable ring device.

The charging station 405 may include a source coil 445 and a resonant coil 440 that are inductively coupled such that a first alternating current on the source coil 445 causes the resonant coil 440 to generate an electromagnetic field. The wearable device 410 may include a load coil 425 that is inductively coupled with the resonant coil 440 such that the electromagnetic field generated by the resonant coil 440 causes the load coil 425 to generate a second alternating current that can be used to charge a battery of the wearable device 410. The load coil 425 and the resonant coil 440 may be configured with the same or similar resonant frequencies to increase the efficiency of power transfer and to reduce coil-misalignment susceptibility. Similarly, the source coil 445 and the resonant coil 440 may be configured with the same or similar resonant frequencies.

The wearable device 410 may include an inner surface 435 (e.g., a curved surface) that is configured to interface with the skin of a user and may include an outer surface 430 (e.g., a curved surface) that is opposite the inner surface 435. The curved surface may be uniformly or non-uniformly curved, and may be an example of, but not limited to, a circular surface, a circumferential surface, and the like. The inner surface 435 may have a radial dimension of RI and the outer surface may have a radial dimension R3. The radial dimension may be an example of a dimension that is measured from an inner and/or center point radially outward. The load coil 425, which may include one or more contiguous loops having a constant radial dimension R2, may be disposed between the inner surface 435 and the outer surface 430 such that the radial dimension R2 of the load coil 425 is between the radial dimension R1 and the radial dimension R3 (where radial dimension R3 is greater than radial dimension R1). That is, the radial dimension R2 of the load coil 425 may be greater than the radial dimension R1 and may be less than the radial dimension R3.

Although shown disposed along the rim surface 420-a for ease of illustration, the load coil 425 may be disposed along the rim surface 420-b so that the load coil 425 can inductively couple with the resonant coil 440 of the charging station 405 when the rim surface 420-b rests on (e.g., physically touches) the charging surface 455. In some examples, there may be a second load coil on the rim surface opposite the rim surface along which the load coil 425 is disposed (e.g., so that the wearable device 410 can charge regardless of the orientation of the wearable device 410 in the charging station 405). The load coil 425 and the second load coil may be combined to form a two-turn load coil, may be combined in parallel, or may be separated. In any event, the second load coil may be configured to inductively couple with the resonant coil 440 and may be physically coupled with a rectifier (e.g., the same rectifier as the load coil 425 or a different rectifier).

The wearable device 410 may also include a rim surface 420-a (e.g., a top edge surface) and a rim surface 420-b (e.g., a bottom edge surface). The inner surface 435, the outer surface 430, the rim surface 420-a, and the rim surface 420-b may be any combination of materials, including metallic materials and non-metallic (e.g., dielectric) materials. For example, one or more of the inner surface 435, the outer surface 430, the rim surface 420-a, and the rim surface 420-b may be a non-metallic material (e.g., a dielectric material such as an epoxy). In such examples, the wearable device 410 may omit the trenches 415.

In some examples (e.g., if each of the inner surface 435, the outer surface 430, the rim surface 420-a, and the rim surface 420-b are metallic), the wearable device 410 may include one or more trenches 415 (e.g., slits, cuts) to reduce or mitigate eddy current phenomenon that might otherwise arise due to inductive resonant charging. For instance, the wearable device 410 may include a trench 415-a that partially or completely cuts through the outer surface 430 and that spans from rim surface 420-a to rim surface 420-b. Additionally or alternatively, the wearable device 410 may include a trench 415-b that partially or completely cuts through the inner surface 435 and that spans from rim surface 420-a to rim surface 420-b. So, the trenches 415 may span the widths of the inner and outer surfaces of the wearable device 410. A trench 415 through a surface may physically separate one end of the surface from another end of the surface. In the case where both the inner surface 435 and the outer surface 430 are metallic, the wearable device 410 may omit the trenches 415 if one or both of the rim surface 420-a and the rim surface 420-b is non-metallic.

In some examples (e.g., if the surfaces of the wearable device 410 are metallic), the wearable device 410 may omit the load coil 425. In such examples, the metallic outer surface 430 of the wearable device 410 (e.g., with the trench 415-a) may function as the load coil and may be inductively coupled with the resonant coil 440 such that the electromagnetic field generated by the resonant coil 440 causes the metallic outer surface 430 to generate a second alternating current that can be used to charge a battery of the wearable device 410. The metallic outer surface 430 and the resonant coil 440 may be configured with the same or similar resonant frequencies to increase the efficiency of power transfer and to reduce coil-misalignment susceptibility. The metallic outer surface 430 can inductively couple with the resonant coil 440 of the charging station 405 when the rim surface 420-b rests on (e.g., physically touches) the charging surface 455 (e.g., so that the wearable device 410 can charge regardless of the orientation of the wearable device 410 in the charging station 405).

In some cases, (e.g., if the surfaces of the wearable device 410 are metallic), the wearable device 410 may omit the load coil 425, and the metallic inner surface 435 of the wearable device 410 may function as the load coil. The metallic inner surface 435 may be inductively coupled with the resonant coil 440 such that the electromagnetic field generated by the resonant coil 440 causes the metallic inner surface 435 to generate a second alternating current that can be used to charge a battery of the wearable device 410. The metallic inner surface 435 and the resonant coil 440 may be configured with the same or similar resonant frequencies to increase the efficiency of power transfer and to reduce coil-misalignment susceptibility. The metallic inner surface 435 can inductively couple with the resonant coil 440 of the charging station 405 when an inner rim surface opposite of the rim surface 420-b rests on (e.g., physically touches) the charging surface 455 (e.g., so that the wearable device 410 can charge regardless of the orientation of the wearable device 410 in the charging station 405).

In some examples, both the metallic outer surface 430 of the wearable device 410 (e.g., with the trench 415-a) and the metallic inner surface 435 of the wearable device 410 may function as the load coil such that more than one load coil may be included in the wearable device 410. Omitting the load coil 425 on the wearable device 410 may reduce the amount of space consumed by coils on the wearable device 410, may reduce manufacturing costs, among other advantages.

The charging station 405 may include a charging surface 455 (also referred to as a support surface or other suitable terminology) that is configured to physically support the wearable device 410. The resonant coil 440 may be below the charging surface 455 (or on the charging surface 455) and above the source coil 445. Thus, the resonant coil 440 may be disposed between the charging surface 455 and the source coil 445. In some examples, the source coil 445 may surround a ground plane 450. The resonant coil 440 may be configured as a planar spiral coil with loops (e.g., turns) that have varying radii (e.g., progressively larger radii). Thus, the resonant coil 440 may be said to expand radially.

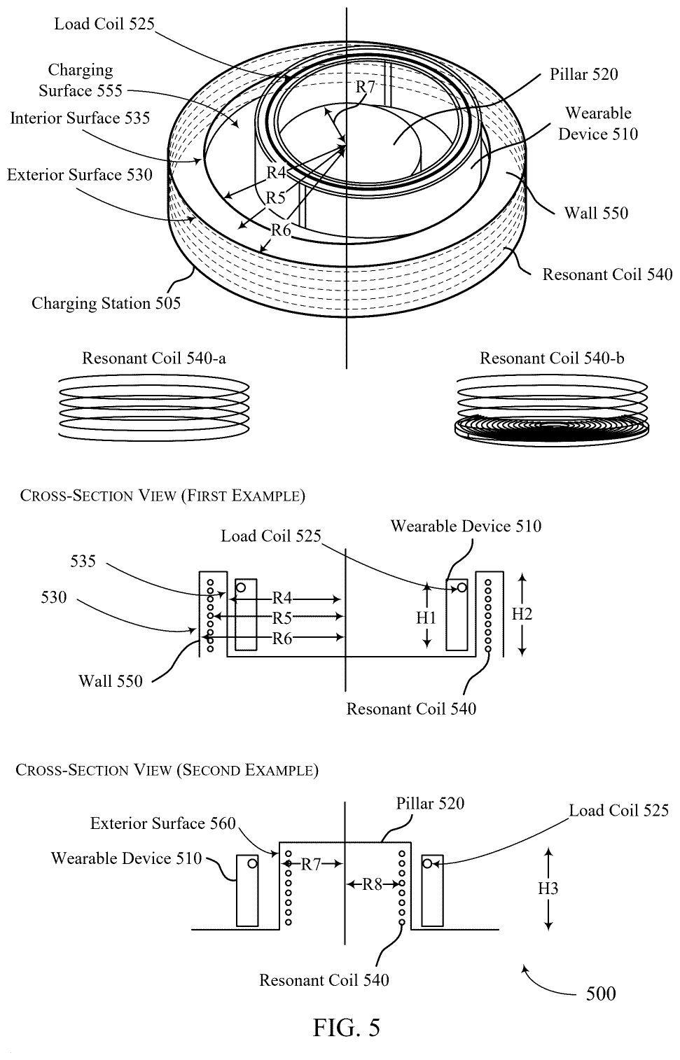

FIG. 5 shows an example of a charging system 500 that supports wireless charging for a wearable device in accordance with aspects of the present disclosure. The charging system 500 may be an example of a charging system 300 or a charging system 400 as described with reference to FIGS. 3 and 4. The charging system may include a charging station 505 and a wearable device 510, which may be a wearable ring device. The charging station 505 may include a wall 550 configured to at least partially surround the wearable device 510, a pillar 520 configured to be at least partially surrounded by the wearable device 510, or both. The wall 550 may have a height H2 that is greater than, equal to, or less than the height of the H1 of the wearable device 510. The pillar 520 may have a height H3 that is greater than, equal to, or less than the height of the H1 of the wearable device 510.

The charging station 505 may include a source coil and a resonant coil 540 that are inductively coupled such that a first alternating current on the source coil causes the resonant coil 540 to generate an electromagnetic field. The wearable device 510 may include a load coil 525 that is inductively coupled with the resonant coil 540 such that the electromagnetic field generated by the resonant coil 540 causes the load coil 525 to generate a second alternating current that can be used to charge a battery of the wearable device 510. The load coil 525 and the resonant coil 540 may be configured with the same or similar resonant frequencies to increase the efficiency of power transfer and to reduce coil-misalignment susceptibility.

The resonant coil 540 may include a cylindrical (spring-like) portion that is standalone, as illustrated by resonant coil 540-a, or that is stacked on and coupled with a planar (disk-like) portion, as illustrated by resonant coil 540-b. Thus, in some examples, the resonant coil 540 (e.g., resonant coil 540-b) may include a combination of a cylindrical coil and a planar coil. The radial dimension of the loops in the cylindrical portion of the resonant coil 540 may be constant whereas the radial dimension of the loops in the planar portion (if present) may vary so that the loops of the planar portion are concentric.

In a first example, at least a portion of the resonant coil 540 (e.g., the cylindrical portion) may be disposed within the wall 550 of the charging station 505. In a second example, at least a portion of the resonant coil 540 (e.g., the cylindrical portion) may be disposed within the pillar 520 of the charging station 505. In either example, the cylindrical portion of the resonant coil 540 may be a spring-like coil that includes loops (e.g., turns) that have a constant radial dimension (e.g., radial dimension R5 for the first example, radial dimension R8 for the second example). If resonant coil 540-b is used, the planar portion may be parallel to the charging surface 555 and may be disposed on the charging surface 555 or beneath the charging surface 555.

In the first example, the cylindrical portion of the resonant coil 540 may be disposed within the wall 550 of the charging station 505. The wall 550 may extend from the charging surface 555 and may at least partially surround (e.g., envelope, encircle) the wearable device 510. The wall 550 may have an exterior surface 530 and an interior surface 535. The radial dimension of the interior surface 535 may be R4 and the radial dimension of the exterior surface 530 may be R6. The cylindrical portion of the resonant coil 540, which include one or more contiguous loops having a constant radial dimension R5, may be disposed between the interior surface 535 and the exterior surface 530 such that the radial dimension R5 of the cylindrical portion of the resonant coil 540 is between the radial dimension R4 and the radial dimension R6. That is, the radial dimension R5 of the cylindrical portion of the resonant coil 540 may be greater than the radial dimension R4 and may be less than the radial dimension R6.

A cross section view of the first example is illustrated in the middle figure. Although illustrated with a single load coil 525, the wearable device 510 may include multiple load coils 525. Although illustrated with the load coil 525 at the top rim surface of the wearable device 510, the load coil 525 may be disposed at the bottom rim surface or anywhere in between the top rim surface and the bottom rim surface. In the first example, the load coil 525 may be closer to an outer surface of the wearable device 510 than to an inner surface of the wearable device 510 (e.g., to improve inductive coupling with the resonant coil 540). In the first example, the source coil may be disposed on or under the charging surface 555, within the wall 550, or elsewhere in the charging station 505.

In the second example, the cylindrical portion of the resonant coil 540 may be disposed within the pillar 520 of the charging station 505. The pillar 520 may extend from the charging surface 555 and may be configured to be at least partially surrounded (e.g., enveloped by, encircled by) the wearable device 510. The pillar 520 may have an exterior surface 560 that has a radial dimension R7. The cylindrical portion of the resonant coil 540, which include one or more contiguous loops having a constant radial dimension R8, may be disposed within the pillar 520. Thus, the radial dimension R8 of the cylindrical portion of the resonant coil 540 may be less than the radial dimension R7 of the pillar 520.

A cross section view of the second example is illustrated in the bottom figure. Although illustrated with a single load coil 525, the wearable device 510 may include multiple load coils 525. Although illustrated with the load coil 525 at the top rim surface of the wearable device 510, the load coil 525 may be disposed at the bottom rim surface or anywhere in between the top rim surface and the bottom rim surface. In the second example, the load coil 525 may be closer to an inner surface of the wearable device 510 than to an outer surface of the wearable device 510 (e.g., to improve inductive coupling with the resonant coil 540). In the second example, the source coil may be disposed on or under the charging surface 555, within the pillar 520, or elsewhere in the charging station 505.

Thus, the charging station 505 may support different configurations of the resonant coil 540 and the load coil 525.

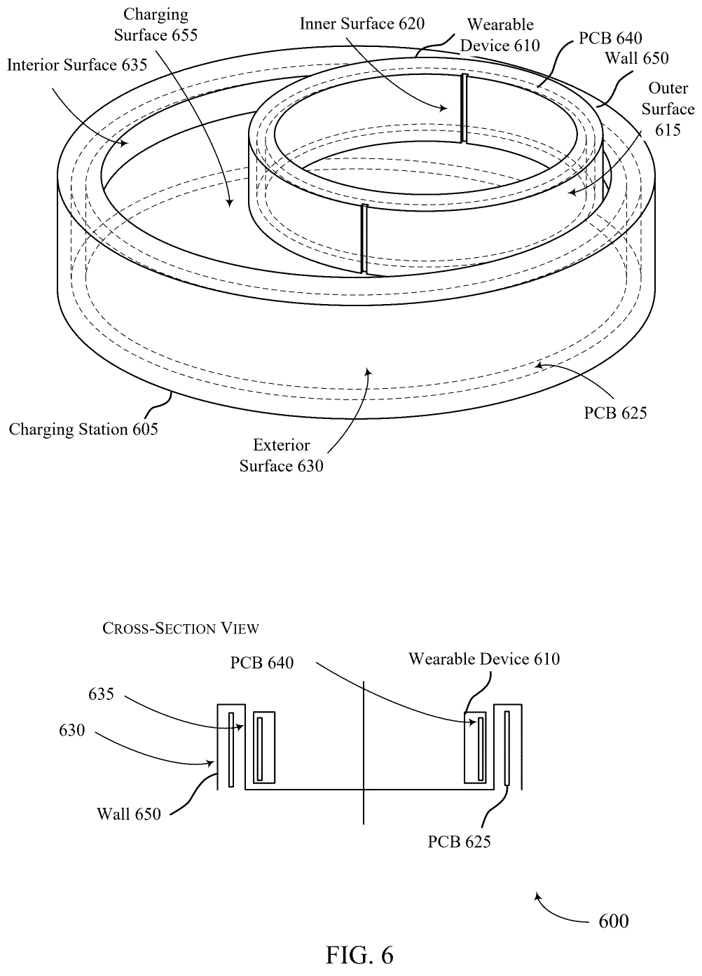

FIG. 6 shows an example of a charging system 600 that supports wireless charging for a wearable device in accordance with aspects of the present disclosure. The charging system 600 may be an example of a charging system 300-a, 300-b, charging system 400, or a charging system 500 as described with reference to FIGS. 3 through 5. The charging system may include a charging station 605 and a wearable device 610, which may be a wearable ring device. The charging station 605 may include a wall 650 configured to at least partially surround the wearable device 610.

In some examples, the charging station 605 may omit a dedicated source coil. In such examples, the flexible printed circuit board (PCB) 625 of the charging station 605 may function as the source coil. In such cases, the charging station 605 may include the PCB 625 and a resonant coil that are inductively coupled such that a first alternating current on the PCB 625 causes the resonant coil to generate an electromagnetic field.

In some examples, the PCB 625 may be disposed within the wall 650 of the charging station 605. The wall 650 may extend from the charging surface 655 and may at least partially surround (e.g., envelope, encircle) the wearable device 610. The wall 650 may have an exterior surface 630 and an interior surface 635. The PCB 625 may be disposed between the interior surface 635 and the exterior surface 630. In such cases, the PCB 625 may be dispersed partially or fully around the perimeter of the charging station 605. Although illustrated with the PCB 625 extending around the entire charging station 605, the PCB 625 may extend along a portion of the perimeter of the charging station 605. Omitting a dedicated source coil on the charging station 605 may reduce the amount of space consumed by coils on the charging station 605, reduce manufacturing cost, among other advantages.

In some examples, the wearable device 610 may omit a dedicated load coil. In such examples, the PCB 640 of the wearable device 610 may function as the load coil and may be inductively coupled with the resonant coil such that the electromagnetic field generated by the resonant coil causes the PCB 640 to generate a second alternating current that can be used to charge a battery of the wearable device 610. The PCB 640 and the resonant coil may be configured with the same or similar resonant frequencies to increase the efficiency of power transfer and to reduce coil-misalignment susceptibility. The PCB 640 can inductively couple with the resonant coil of the charging station 605 when a rim surface of the wearable device 610 rests on (e.g., physically touches) the charging surface 655 (e.g., so that the wearable device 610 can charge regardless of the orientation of the wearable device 610 in the charging station 605).

In some examples, the PCB 640 may be disposed between the inner surface 620 and the outer surface 615 of the wearable device 610. In such cases, the PCB 640 may be dispersed around the perimeter of the wearable device 610. Although illustrated with the PCB 640 extending around the entire wearable device 610, the PCB 640 may extend along a portion of the perimeter of the wearable device 610. Omitting a dedicated load coil on the wearable device 610 may reduce the amount of space consumed by coils on the wearable device 610, reduce manufacturing costs, among other advantages.

In some examples, a dedicated source coil may be omitted from the charging station 605 (e.g., the PCB 625 may function as the source coil) while the wearable device 610 may include a dedicated load coil, as described with reference to FIGS. 3 through 5. In other examples, a dedicated load coil may be omitted from the wearable device 610 (e.g., the PCB 640 may function as the load coil) while the charging station may include a dedicated source coil, as described with reference to FIGS. 3 through 5.

As shown in the cross-section view of FIG. 6, the PCB 625 may align with the PCB 640, thereby improving inductive coupling with the resonant coil. Thus, the charging station 605 may support different configurations of the resonant coil, the PCB 625, and the PCB 640.

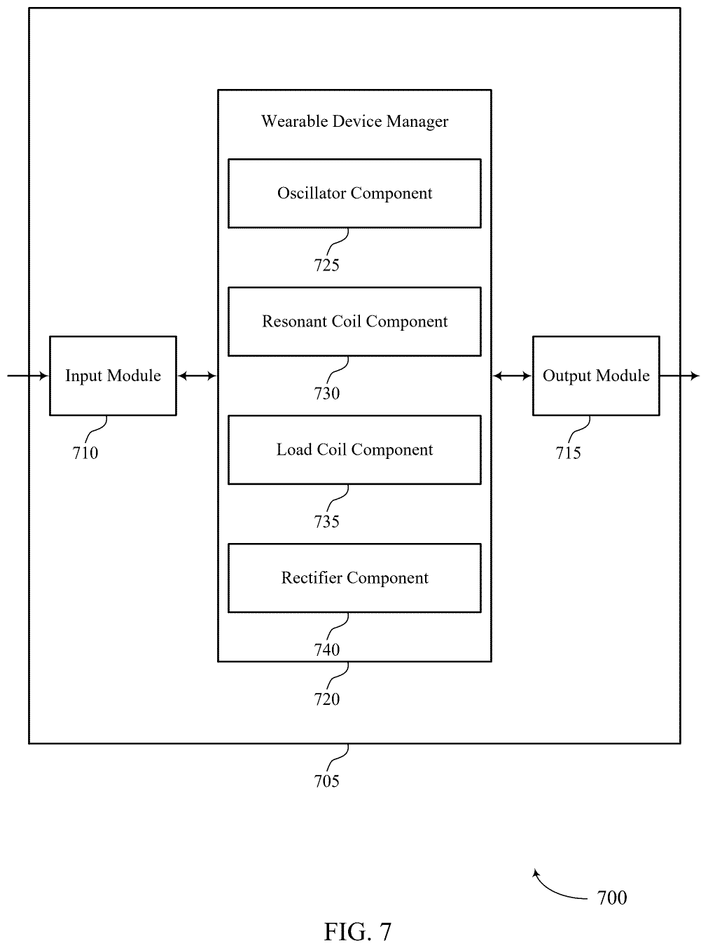

FIG. 7 shows a block diagram 700 of a device 705 that supports wireless charging for a wearable device in accordance with aspects of the present disclosure. The device 705 may include an input module 710, an output module 715, and a wearable device manager 720. The device 705, or one of more components of the device 705 (e.g., the input module 710, the output module 715, the wearable device manager 720), may include at least one processor, which may be coupled with at least one memory, to support the described techniques. Each of these components may be in communication with one another (e.g., via one or more buses).

For example, the wearable device manager 720 may include an oscillator component 725, a resonant coil component 730, a load coil component 735, a rectifier component 740, or any combination thereof. In some examples, the wearable device manager 720, or various components thereof, may be configured to perform various operations (e.g., receiving, monitoring, transmitting) using or otherwise in cooperation with the input module 710, the output module 715, or both. For example, the wearable device manager 720 may receive information from the input module 710, send information to the output module 715, or be integrated in combination with the input module 710, the output module 715, or both to receive information, transmit information, or perform various other operations as described herein.

The oscillator component 725 may be configured as or otherwise support a means for generating, by an oscillator of a charging station, a first alternating electrical current on a resonant coil or a source coil. The resonant coil component 730 may be configured as or otherwise support a means for generating, by the resonant coil of the charging station, an electromagnetic field based at least in part on the first alternating electrical current. The load coil component 735 may be configured as or otherwise support a means for generating, by a load coil inductively coupled with the resonant coil of the charging station, a second alternating current based at least in part on the electromagnetic field generated by the resonant coil, the load coil included in a wearable ring device and configured to have a same resonant frequency as the resonant coil of the charging station. The rectifier component 740 may be configured as or otherwise support a means for rectifying, by a rectifier physically coupled with the load coil, the second alternating current.

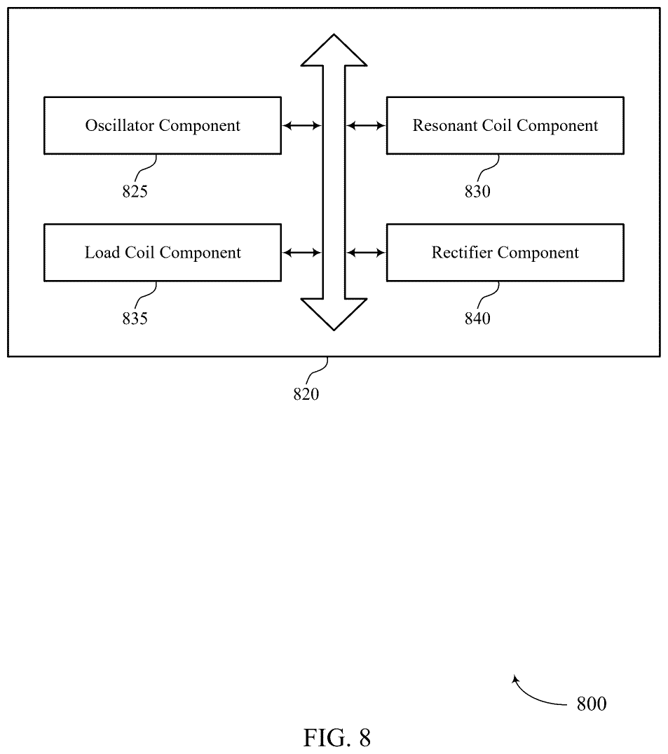

FIG. 8 shows a block diagram 800 of a wearable device manager 820 that supports wireless charging for a wearable device in accordance with aspects of the present disclosure. The wearable device manager 820 may be an example of aspects of a wearable device manager or a wearable device manager 720, or both, as described herein. The wearable device manager 820, or various components thereof, may be an example of means for performing various aspects of wireless charging for a wearable device as described herein. For example, the wearable device manager 820 may include an oscillator component 825, a resonant coil component 830, a load coil component 835, a rectifier component 840, or any combination thereof. Each of these components, or components of subcomponents thereof (e.g., one or more processors, one or more memories), may communicate, directly or indirectly, with one another (e.g., via one or more buses).

The oscillator component 825 may be configured as or otherwise support a means for generating, by an oscillator of a charging station, a first alternating electrical current on a resonant coil or a source coil. The resonant coil component 830 may be configured as or otherwise support a means for generating, by the resonant coil of the charging station, an electromagnetic field based at least in part on the first alternating electrical current. The load coil component 835 may be configured as or otherwise support a means for generating, by a load coil inductively coupled with the resonant coil of the charging station, a second alternating current based at least in part on the electromagnetic field generated by the resonant coil, the load coil included in a wearable ring device and configured to have a same resonant frequency as the resonant coil of the charging station. The rectifier component 840 may be configured as or otherwise support a means for rectifying, by a rectifier physically coupled with the load coil, the second alternating current.

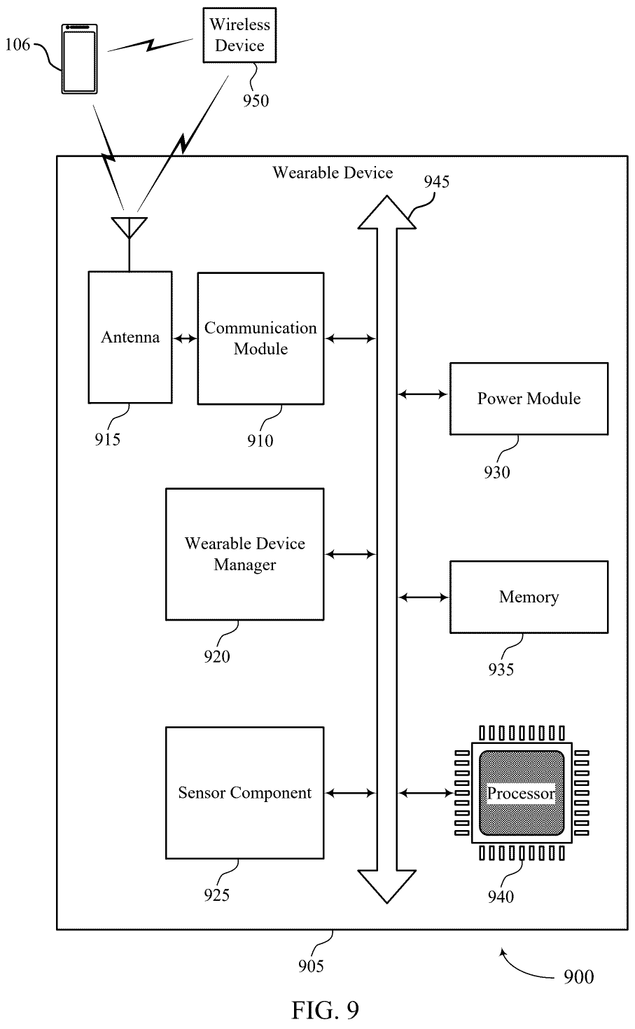

FIG. 9 shows a diagram of a system 900 including a device 905 that supports wireless charging for a wearable device in accordance with aspects of the present disclosure. The device 905 may be an example of or include components of a device 705 as described herein. The device 905 may include an example of a wearable device 104, as described previously herein. The device 905 may include components for bi-directional communications including components for transmitting and receiving communications with a user device 106 and a server 110, such as a wearable device manager 920, a communication module 910, one or more antennas 915, a sensor component 925, a power module 930, at least one memory 935, at least one processor 940, and a wireless device 950. These components may be in electronic communication or otherwise coupled (e.g., operatively, communicatively, functionally, electronically, electrically) via one or more buses (e.g., a bus 945).

For example, the wearable device manager 920 may be configured as or otherwise support a means for generating, by an oscillator of a charging station, a first alternating electrical current on a resonant coil or a source coil. The wearable device manager 920 may be configured as or otherwise support a means for generating, by the resonant coil of the charging station, an electromagnetic field based at least in part on the first alternating electrical current. The wearable device manager 920 may be configured as or otherwise support a means for generating, by a load coil inductively coupled with the resonant coil of the charging station, a second alternating current based at least in part on the electromagnetic field generated by the resonant coil, the load coil included in a wearable ring device and configured to have a same resonant frequency as the resonant coil of the charging station. The wearable device manager 920 may be configured as or otherwise support a means for rectifying, by a rectifier physically coupling with the load coil, the second alternating current.

By including or configuring the wearable device manager 920 in accordance with examples as described herein, the device 905 may support techniques for improved wireless charging.

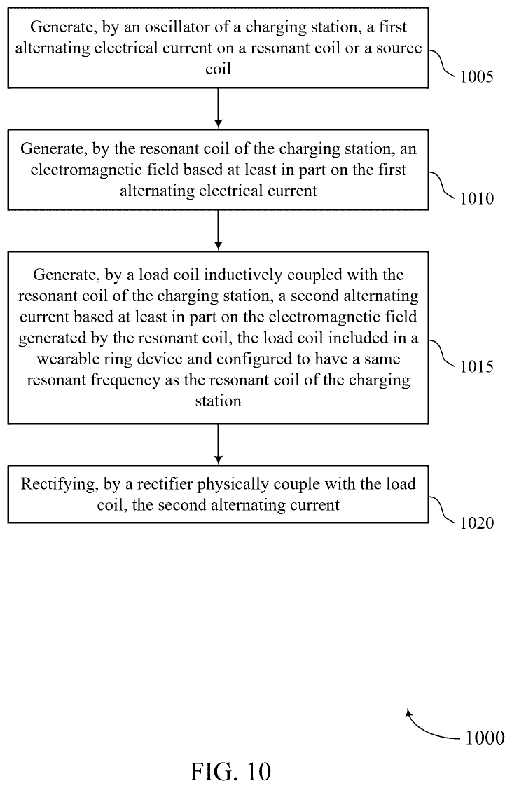

FIG. 10 shows a flowchart illustrating a method 1000 that supports wireless charging for a wearable device in accordance with aspects of the present disclosure. The operations of the method 1000 may be implemented by a wearable device or its components as described herein. For example, the operations of the method 1000 may be performed by a wearable device as described with reference to FIGS. 1 through 9. In some examples, a wearable device may execute a set of instructions to control the functional elements of the wearable device to perform the described functions. Additionally, or alternatively, the wearable device may perform aspects of the described functions using special-purpose hardware.

At 1005, the method may include generating, by an oscillator of a charging station, a first alternating electrical current on a resonant coil or a source coil. The operations of 1005 may be performed in accordance with examples as disclosed herein. In some examples, aspects of the operations of 1005 may be performed by an oscillator component 825 as described with reference to FIG. 8.

At 1010, the method may include generating, by the resonant coil of the charging station, an electromagnetic field based at least in part on the first alternating electrical current. The operations of 1010 may be performed in accordance with examples as disclosed herein. In some examples, aspects of the operations of 1010 may be performed by a resonant coil component 830 as described with reference to FIG. 8.

At 1015, the method may include generating, by a load coil inductively coupled with the resonant coil of the charging station, a second alternating current based at least in part on the electromagnetic field generated by the resonant coil, the load coil included in a wearable ring device and configured to have a same resonant frequency as the resonant coil of the charging station. The operations of 1015 may be performed in accordance with examples as disclosed herein. In some examples, aspects of the operations of 1015 may be performed by a load coil component 835 as described with reference to FIG. 8.

At 1020, the method may include rectifying, by a rectifier physically coupled with the load coil, the second alternating current. The operations of 1020 may be performed in accordance with examples as disclosed herein. In some examples, aspects of the operations of 1020 may be performed by a rectifier component 840 as described with reference to FIG. 8.

It should be noted that the methods described above describe possible implementations, and that the operations and the steps may be rearranged or otherwise modified and that other implementations are possible. Furthermore, aspects from two or more of the methods may be combined.

A method by an apparatus is described. The method may include generating, by an oscillator of a charging station, a first alternating electrical current on a resonant coil or a source coil, generating, by the resonant coil of the charging station, an electromagnetic field based at least in part on the first alternating electrical current, generating, by a load coil inductively coupled with the resonant coil of the charging station, a second alternating current based at least in part on the electromagnetic field generated by the resonant coil, the load coil included in a wearable ring device and configured to have a same resonant frequency as the resonant coil of the charging station, and rectifying, by a rectifier physically coupled with the load coil, the second alternating current.

An apparatus is described. The apparatus may include one or more memories storing processor executable code, and one or more processors coupled with the one or more memories. The one or more processors may individually or collectively be operable to execute the code to cause the apparatus to generate, by an oscillator of a charging station, a first alternating electrical current on a resonant coil or a source coil, generate, by the resonant coil of the charging station, an electromagnetic field based at least in part on the first alternating electrical current, generate, by a load coil inductively coupled with the resonant coil of the charging station, a second alternating current based at least in part on the electromagnetic field generated by the resonant coil, the load coil included in a wearable ring device and configured to have a same resonant frequency as the resonant coil of the charging station, and rectifying, by a rectifier physically couple with the load coil, the second alternating current.

Another apparatus is described. The apparatus may include means for generating, by an oscillator of a charging station, a first alternating electrical current on a resonant coil or a source coil, means for generating, by the resonant coil of the charging station, an electromagnetic field based at least in part on the first alternating electrical current, means for generating, by a load coil inductively coupled with the resonant coil of the charging station, a second alternating current based at least in part on the electromagnetic field generated by the resonant coil, the load coil included in a wearable ring device and configured to have a same resonant frequency as the resonant coil of the charging station, and means for rectifying, by a rectifier physically coupled with the load coil, the second alternating current.

A non-transitory computer-readable medium storing code is described. The code may include instructions executable by one or more processors to generate, by an oscillator of a charging station, a first alternating electrical current on a resonant coil or a source coil, generate, by the resonant coil of the charging station, an electromagnetic field based at least in part on the first alternating electrical current, generate, by a load coil inductively coupled with the resonant coil of the charging station, a second alternating current based at least in part on the electromagnetic field generated by the resonant coil, the load coil included in a wearable ring device and configured to have a same resonant frequency as the resonant coil of the charging station, and rectifying, by a rectifier physically couple with the load coil, the second alternating current.

An apparatus is described. The apparatus may include a charging station comprising, a resonant coil coupled with an oscillator, wherein the oscillator is configured to generate a first alternating current on the resonant coil or on a source coil, and wherein the resonant coil is configured to generate an electromagnetic field based at least in part on the first alternating current, a wearable ring device comprising, a load coil inductively coupled with the resonant coil of the charging station, wherein the load coil is configured to have a same resonant frequency as the resonant coil, and wherein the load coil is configured to generate a second alternating current based at least in part on the electromagnetic field generated by the resonant coil, and a rectifier physically coupled with the load coil and configured to rectify the second alternating current.

In some examples of the apparatus, the charging station comprises the source coil, the source coil may be physically coupled with the oscillator, and the resonant coil may be inductively coupled with the source coil.

In some examples of the apparatus, the wearable ring device further comprises a trench through the metallic outer surface of the wearable ring device, the trench spanning from a first curved surface of the wearable ring device to a second curved surface of the wearable ring device.

In some examples of the apparatus, the inner surface comprises a metallic inner surface.

In some examples of the apparatus, the metallic inner surface, the metallic outer surface, or both comprises the load coil.

In some examples of the apparatus, the wearable ring device comprises a flexible printed circuit board positioned between the metallic outer surface and the inner surface.

In some examples of the apparatus, the flexible printed circuit board comprises the load coil.

Some examples of the apparatus may further include a trench through the metallic inner surface of the wearable ring device, the trench spanning from a first curved surface of the wearable ring device to a second curved surface of the wearable ring device.

In some examples of the apparatus, the wearable ring device comprises a metallic outer surface and a non-metallic curved surface.

In some examples of the apparatus, the wearable ring device comprises a non-metallic outer surface opposite a non-metallic inner surface.

In some examples of the apparatus, the load coil comprises at least one contiguous loop having a first radial dimension that may be between a second radial dimension of an outer surface of the wearable ring device and a third radial dimension of an inner surface of the wearable ring device.

In some examples of the apparatus, the load coil may be disposed along a first curved edge of the wearable ring device.

In some examples of the apparatus, the wearable ring device comprises a second load coil that may be disposed along a second curved edge of the wearable ring device and that may be configured to inductively couple with the resonant coil of the charging station, the second load coil physically coupled with the rectifier and configured to may have the same resonant frequency as the resonant coil.

In some examples of the apparatus, the charging station comprises a surface configured to support the wearable ring device and the resonant coil comprises a planar spiral coil and may be disposed between the source coil and the surface of the charging station.

In some examples of the apparatus, the resonant coil comprises a planar coil portion that may be parallel to the surface and a cylindrical coil portion configured to at least partially surround the wearable ring device.

In some examples of the apparatus, the charging station comprises a surface configured to support the wearable ring device and an outer wall configured to at least partially surround the wearable ring device and the resonant coil may be disposed within the outer wall.

In some examples of the apparatus, the charging station comprises a flexible printed circuit board disposed within the outer wall and the flexible printed circuit board comprises the source coil.

In some examples of the apparatus, the resonant coil may have a first radial dimension that may be between a second radial dimension of an outer surface of the outer wall and a third radial dimension of an inner surface of the outer wall.

In some examples of the apparatus, the charging station comprises a surface configured to support the wearable ring device and a pillar which the wearable ring device may be configured to at least partially surround and the resonant coil may be disposed within the pillar.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A charging system, comprising:
a charging station comprising:
a resonant coil coupled with an oscillator, wherein the oscillator is configured to generate a first alternating current on the resonant coil or on a source coil, and wherein the resonant coil is configured to generate an electromagnetic field based at least in part on the first alternating current; and
a wearable ring device comprising:
a load coil inductively coupled with the resonant coil of the charging station, wherein the load coil is configured to have a same resonant frequency as the resonant coil, and wherein the load coil is configured to generate a second alternating current based at least in part on the electromagnetic field generated by the resonant coil; and
a rectifier physically coupled with the load coil and configured to rectify the second alternating current.

2. The charging system of claim 1, wherein the charging station comprises the source coil, wherein the source coil is physically coupled with the oscillator, and wherein the resonant coil is inductively coupled with the source coil.

3. The charging system of claim 1, wherein the wearable ring device comprises a metallic outer surface opposite an inner surface, wherein the wearable ring device further comprises:
a trench through the metallic outer surface of the wearable ring device, the trench spanning from a first curved surface of the wearable ring device to a second curved surface of the wearable ring device.

4. The charging system of claim 3, wherein the inner surface comprises a metallic inner surface.

5. The charging system of claim 4, wherein the metallic inner surface, the metallic outer surface, or both comprises the load coil.

6. The charging system of claim 3, wherein the wearable ring device comprises a flexible printed circuit board positioned between the metallic outer surface and the inner surface.

7. The charging system of claim 6, wherein the flexible printed circuit board comprises the load coil.

8. The charging system of claim 1, wherein the wearable ring device comprises a metallic inner surface, the wearable ring device further comprising:
a trench through the metallic inner surface of the wearable ring device, the trench spanning from a first curved surface of the wearable ring device to a second curved surface of the wearable ring device.

9. The charging system of claim 1, wherein the wearable ring device comprises a metallic outer surface and a non-metallic curved surface.

10. The charging system of claim 1, wherein the wearable ring device comprises a non-metallic outer surface opposite a non-metallic inner surface.

11. The charging system of claim 1, wherein the load coil comprises at least one contiguous loop having a first radial dimension that is between a second radial dimension of an outer surface of the wearable ring device and a third radial dimension of an inner surface of the wearable ring device.

12. The charging system of claim 11, wherein the load coil is disposed along a first curved edge of the wearable ring device.

13. The charging system of claim 12, wherein the wearable ring device comprises a second load coil that is disposed along a second curved edge of the wearable ring device and that is configured to inductively couple with the resonant coil of the charging station, the second load coil physically coupled with the rectifier and configured to have the same resonant frequency as the resonant coil.

14. The charging system of claim 1, wherein the charging station comprises a surface configured to support the wearable ring device, and wherein the resonant coil comprises a planar spiral coil and is disposed between the source coil and the surface of the charging station.

15. The charging system of claim 1, wherein the charging station comprises a surface configured to support the wearable ring device, and wherein the resonant coil comprises:
a planar coil portion that is parallel to the surface, and
a cylindrical coil portion configured to at least partially surround the wearable ring device.

16. The charging system of claim 1, wherein the charging station comprises a surface configured to support the wearable ring device and an outer wall configured to at least partially surround the wearable ring device, and wherein the resonant coil is disposed within the outer wall.

17. The charging system of claim 16, wherein the charging station comprises a flexible printed circuit board disposed within the outer wall, and wherein the flexible printed circuit board comprises the source coil.

18. The charging system of claim 16, wherein the resonant coil has a first radial dimension that is between a second radial dimension of an outer surface of the outer wall and a third radial dimension of an inner surface of the outer wall.

19. The charging system of claim 1, wherein the charging station comprises a surface configured to support the wearable ring device and a pillar which the wearable ring device is configured to at least partially surround, and wherein the resonant coil is disposed within the pillar.

20. A method, comprising:
generating, by an oscillator of a charging station, a first alternating electrical current on a resonant coil or a source coil;
generating, by the resonant coil of the charging station, an electromagnetic field based at least in part on the first alternating electrical current;
generating, by a load coil inductively coupled with the resonant coil of the charging station, a second alternating current based at least in part on the electromagnetic field generated by the resonant coil, the load coil included in a wearable ring device and configured to have a same resonant frequency as the resonant coil of the charging station; and
rectifying, by a rectifier physically coupled with the load coil, the second alternating current.
